(19) United States
(12) Patent Application Publication
(10) Pub. No.: US 2013/0183323 A1
(43) Pub. Date: Jul. 18, 2013

(54) TARGETED ANTIBIOTIC AND ANTIMICROBIAL TREATMENTS FOR PERSONALIZED ADMINISTRATION

(71) Applicant: Hua Wang, Columbus, OH (US)

(72) Inventor: Hua Wang, Columbus, OH (US)

(21) Appl. No.: 13/720,934

(22) Filed: Dec. 19, 2012

Related U.S. Application Data
(60) Provisional application No. 61/577,242, filed on Dec. 19, 2011.

(51) Int. Cl. A61K 47/48

(52) U.S. Cl. CPC A61K 47/48369
USPC 424/178.1

(57) ABSTRACT

A solution for the bottleneck issues in antibiotic treatment is to use novel antibiotic formulas with targeted delivery customized based on the nature of the infection and resistance profile of the infectious agent(s).

### CROSS-REFERENCE TO RELATED APPLICATIONS

The application is a utility patent converting from provisional patent with Application No. 61/577,242 (EFS ID 11649468), Confirmation Number 8500, filed by Lynda M. Fitzpatrick/Tracy Bruesewitz, with Attorney Docket Number 029784-9007-US00, on 19-DEC-2011, for Inventor/Applicant Hua Wang from the Ohio State University. The Institute has recently assigned the rights to the inventor Hua Wang.

### BACKGROUND

Antibiotic resistance has been a major public health issue. In the past it was believed the use of antibiotics was the main reason for resistance development, and thus the main control strategy has been to minimize the applications of antibiotics in both human clinical therapy and food animal production. However, this strategy likely is mistargeted. In fact, limiting the use of antibiotics could contribute to more health problems such as the development of biofilm-related antibiotic treatment-persistent infection; persistent inflammation can trigger additional health problems. Delayed treatment for upper respiratory infections, even those initially triggered by a virus but followed with secondary bacterial infection, likely has been one reason for the development of persistent situations such as asthma, prevalent in children and adults today.

Several contributors to the resistance problem include: A) antibiotics administered in an inappropriate format such that a significant amount of the drug occurs in the gastrointestinal tract causing resistance in gut microflora, and then both the drug, derivatives and antibiotic resistant gut bacteria are excreted in the feces/manure or urine for some drug residues or derivatives, which further impacts the environment, food and hosts; B) non-discriminative exposure of host microflora to the antibiotics as random shots; C) effective dosage not reaching the infection site and being unable to kill target organisms. When administered, the antibiotic is equally distributed in the whole body, despite only the infection site needing the drug. A similar problem exists with drugs for cancer treatment, where major breakthrough for targeted delivery has been achieved.

However unlike cancer treatment, of which the types of problems are limited and defined (i.e., breast, liver, lung, etc), many microbes can cause disease at single (local) or multiple sites (systematic), and each microorganism may have a different resistant spectrum against antibiotics, and all of which can keep changing. Particularly, microorganisms can develop or acquire genetic elements encoding for resistance to any antimicrobials including antibiotics through mutation or horizontal gene transfer events (which may happen in minutes). In addition, resistance to the same drug encoded by the same resistance gene can lead to varied minimum drug inhibitory concentration (MIC) even in same genus or species of bacteria. Many bacteria have developed resistance to multiple drugs, which can lead to failure in treatment. This unpredictability creates a moving target for treating infections caused by antibiotic resistant bacteria in patients. Thus a strategy which works for targeted cancer treatment, or similar strategy intended for bacteria therapy using fixed formula, such as directly linking one antibiotic to an organism-specific antibody, is ineffective to deal with the complicated issues arising from practical treatment of microbial infection varied in resistance profiles in patients.

### SUMMARY

In one aspect, the invention provides a method for targeting antibiotics with the flexibility to 1) an infection site; 2) single or groups of microbes; 3) host responsive molecular targets, suitable for personalized treatment. In another aspect, the invention also provides a method for modifying antibiotics with adjunct(s) for enhanced antimicrobial activities. The method comprises administering to a subject, a personalized antibiotic formula with an antibiotic-ligand conjugate or an array of antibiotic-ligand conjugates. The conjugate contains an antibiotic modified to include a first binding site and a ligand modified to include a second binding site. The first and second binding sites link to form the conjugate directly or through a bridging molecule. Independent lists of antibiotics or antimicrobials and ligands enable unlimited combination for personalized drug formulation and targeted treatment.

### BRIEF DESCRIPTION OF THE DRAWINGS

FIG. 1 includes graph showing tetracycline resistance-encoding gene pool before and after exposure using two drug delivery methods.

### DETAILED DESCRIPTION

Disclosed are methods, compositions and kits for targeting antibiotics to an infection site, specific organism(s) or host responding molecule(s). An antibiotic that includes at a non-active region (a region not required for antibiotic activity) a modification (providing a first binding site) which facilitates binding of a complementary component, is contacted with an antibody or other ligand. The antibody or ligand is modified to include the complementary component (providing a second binding site). The first and second binding sites can serve as part of a clamp pair directly or through a bridging molecule. The conjugate is formed by contacting the modified antibody with the modified antibiotic with or without a bridging molecule, such that binding occurs between the first binding site and second binding site.

Disclosed are novel formulas of antibiotics, such as antibiotic-antibody or antibiotic-adjunct conjugates, which include a simple modification of the antibiotic that facilitates targeted delivery, using a novel, multiple-ingredient composition. The antibody in the antibiotic-antibody conjugate retains the ability to bind to and form a conjugate with, for example, a cell, tissue, organ, organism, microorganism, infectious agent, bacterium, virus, fungus, disease site, or immune response factor, thereby facilitating targeted delivery of the antibiotic.

Component 1 can be an antibiotic with single or multiple structure modifications at non-active site(s) to enable the binding of single or multiple ligand(s). For instance, the antibiotic may be modified to enable the binding of biotin, or nickel, or peptide, or nucleic acid, or any other small element, which can serve as part of a clamp pair, designated ClampP1 (or P1).

Examples of antibiotics and antimicrobials which may be used as Component 1 include but not limit to the following: Sulphonamide (sulfapyridine), beta-lactam (penecillin, cephalosporins, carbapenems and monobactams), bacterial peptide (bacteriocin, polmixin, nisin, pediocin, etc), aminoglycoside (gentamicin, kanamycin, neomycin and streptomycin), Nitrofuran (Nitrofurantoin), Hexamine (Methenamine mandelate), Chloramphenicol (chloramphenicol), Tetracycline (oxytetracycline, tetracycline, Chlortetracycline, demeclocycline, doxycycline and minocycline), Isoriazid (Isoziazid), Viomycin (Viomycin), Microlides/ketolides (Erythromycin, clarithromycin, dirithromycin, telithroymycin), Lincosamide (Lincomycin), Streptogramin (Virginiamycin), Cycloserine (Cycloserine), Glycopeptide (Vancomycin), Novobiocin (Novobiocin), Ansamycin(Rifamycin), Nitroimidazole (Tinidazole), Ethambutol (Ethambutol), Quinolone (Nalidixic acid, Ciprofloxacin, gemifloxacin, moxifloxacin and trovafloxacin), Fusidane (Fusidic acid), Diaminopyrimidine (trimethoprim), Phosphonate (Fosfomycin), Pseudomonic acid (Muprirocin), Oxazolidinone (Linezolid), Lipopeptides (Daptomycin), sulfonamides (sulfamethoxazole, sulfadoxine, sulfasalazine and silver sulfadiazine), Azithromycin (Zithromax), nisin, pediocin, bacteriocins, lantibiotics, its kind or derivatives.

Component 2 can be a ligand or antibody of any targeted infectious agents or commensal microorganisms, or any tissue- or organ-specific markers, or specific host immune response factors, including but not limited to white blood cells, phagocytes, their breakdown derivative markers or other inflammation signals, or adjuncts such as biofilm-fighting agent(s), etc., and linked to a complementary element able to form clamp pair with ClampP1, designated ClampP2 (or P2).

There may optionally be a third component, Component3, which is an intermediate that binds between ClampP1 and ClampP2.

Component1 and 2 can also both be linked to ClampP1 and be connected through intermediate(s) such as ClampP2 with multiple binding sites.

Non-limiting examples of ClampP1/P2 affinity binding pairs include biotin/avidin(streptoavidin), nickel/His tag, divalent ions/chelators, peptide, antibody etc. and other small elements/ligands.

Examples of epitope tags and pairs that can be used as clamps include, but are not limited to, the following: biotin (avidin, streptavidin), GSTs and glutathione, poly His tag, Ni or cobalt or affinity agent(s), natural or synthesized FLAG-tag or FLAG octapeptide (or other peptide), HA (hemagglutinin) tag, myc-tag (N-EQKLISEEDL-C) and antibody (such as 9E10), drug conjugates, multi-specific antibodies, Fc engineered antibodies, scFv fused receptors, peptide(s) and engineered antigens and antibodies, site-specific antibodies and biomarkers; anti-DDDDK tag, anti-R Phycoerythrin, anti-VSV-G tag, anti-digoxigenin, anti-biotin, anti-FITC, anti-poly (5×, 6×, 8× etc) His tag, anti-V5 tag, nucleotides, small molecules with reactive groups, and those commonly used in cancer targeted drug delivery.

ClampP1 and ClampP2 may be switched between Componetl and Component2, or can be the same, especially when a component 3 intermediate is involved.

In cases when P1 or P2 has multiple affinity sites, the conjugate can be achieved by an intermediate between P1 or P2, such as Component1-ClampP1-(intermediate)-ClampP2-Component2, or switching the positions of P1 and P2. Examples such as Biotinylated Component1(antibiotic)-Avidin (Streptoavidin)-biotinylated Component2 (antibody), or when an intermediate P3 can bind to both ClampP1 and ClampP2, can also take a format of Component1P1-P3-P2Component2. In the case of avidin type of molecule, multiple biotinylated molecules (such as 2, 3, 4 of antibiotics and/or target-specific antibodies, etc.) can bind to the intermediate core P3.

Component1 and Component2 can be antibiotics and antibodies, or derivatives having a simple structure modification, with the structure needed to enable the rapid assembly of the conjugated structure in vitro. For example, the structure feature existed or through in vitro biotinylation using commercial kits, which enables additional affinity binding, or other types of covalent or non-covalent affinity binding exemplified at the end of the document.

Component2 serves as the target tracker for infectious agents, infected tissue/organ or immune responses that are gathered or are moving towards the infected sites, so the single or multiple antibiotics will be delivered and hooked to the target.

Component1ClampP1 and Component2ClampP2 can be manufactured and stored as independent ingredients, with numerous possible combinations available for production on demand or on site for personalized treatment, although conjugated final drugs can be made available by manufacturers for common infectious agents.

In addition, adjunct Component3 ClampP2 (or P1) can be an enzyme or active domain thereof able to breakdown biofilm matrices (such as described in U.S. Pat. No. 8,038,990, the entire disclosure of which is hereby incorporated by reference, including proteins, as well as other enzymes for other matrices), or any factors/molecules involved in biofilm formation, regulation or maintenance functions, which can be a supplemental ingredient dedicated to improve treatment efficiency against biofilm cells. It also can be manufactured and kept separately. Component3 can also be an antibiotic enhancer, such as a beta-lactamase inhibitor.

A blocker with just a ClampP1 or P2 or molecule(s) with a similar nature, or simply with an unmodified drug can be used in combination with Component2 to customize antimicrobial drug formulation (the ratio of drug ingredients available to be captured by different targets, as well as for systematic cleanup-antibiotic without target conjugate, if needed) for each patient depending on the range and severity of the disease.

A customized (personalized) antibiotic formula may have one or more antibiotics conjugated to a narrow or wide range (for example, 1, 2, 3, 4, 5, 6, 7, 8, 9 10 or more) of microbial, tissue, organ or immune component target spectrum, for targeted, semi-targeted, or systematic coverage based on treatment needs.

Component1 may be an existing antibiotic or a new antibiotic, or may be an antimicrobial compound such as peptides and lantibiotics.

Component2 may be an antibody or simply a ligand with specificity against the whole cell of any microorganism, particular surface markers of the organisms, or any tissue- or organ-specific markers, or specific host immune response factors, markers or derivatives.

The customized antibiotics are assembled when needed for applications for each patient with any infection, once the infection site and organisms responsible for the infection or located in the same microbial consortium and their antibiotic susceptibility spectrum identified, by mixing a chosen antibiotic(s) (Component1), and the corresponding antibody or antibodies (Component2) with the proper clamp pair, such as in the pharmacy or clinic.

There are numerous possible combinations of Component1 and Component2 having a proper clamp hook.

Single or multiple types of Component1 may be mixed with single or multiple type(s) of Component2, such as one antibody specific for the organism, and another antibody specific for the tissue/organ, with or without supplementing Component3.

The combination may be Component1 with multiple ligand binding sites to bind to different antibodies, or Component1 with different single Component2 (such as organism-specific and tissue/organ-specific antibodies) in a mix, or mediated through an intermediate (such as a biotin-avidin-biotin combination or involving other epitope tags).

The customized antibiotics or antimicrobial compounds may be administered systematically or locally, moved towards and/or trapped/bound to the targeted tissue/organ/microbes with elevated concentration and for extended period of time in vivo.

The above treatment format can significantly increase the local/effective concentration of the antimicrobial compounds in the target site/area and reduce its frequency to be metabolized in the body by the liver or kidney, thereby reducing the overall dosage needed for administration, while maintaining or even increasing the local concentration to achieve effective treatment and reduced resistance development in unrelated microbiota.

The conjugate can be administered within about 1 hour, within about 2 hours, within about 3 hours, within about 4 hours, within about 5 hours, within about 6 hours, within about 7 hours, within about 8 hours, within about 9 hours, within about 10 hours, within about 11 hours, within about 12 hours, within about 15 hours, within about 18 hours, within about 21 hours, within about 24 hours, within about 36 hours, within about 48 hours, within about 3 days, within about 4 days, within about 5 days, within about 6 days, within about 7 days, within about 2 weeks, within about 3 weeks, or within about 2 months of being formed.

The amount of antibiotic needed may less than about 90%, less than about 80%, less than about 70%, less than about 60%, less than about 50%, less than about 40%, less than about 30%, less than about 20%, less than about 10%, less than about 5%, less than about 4%, less than about 3%, less than about 2%, or less than about 1% of the conventional dose given when the antibiotic is administered alone, or higher than the conventional dose but concentrated to specific target(s) thus with increased dosage limits overall, by intravenous, muscle, patch or oral.

The treatment is also applicable to treat drug resistant bacteria, because drug resistant bacteria are just less sensitive to the drug in certain concentration. By increasing local concentration without reaching the lethal level for the host (in the whole body), the resistant bacteria can still be killed or damaged.

This conjugates formed may be used in acute infections, and also many chronic conditions such as certain cancer and inflammatory bowl diseases, joint diseases, etc., triggered by microbes.

The concept is also applicable to drug development for treatments against other microbes, including but not limiting to molds, yeasts, virus, etc. and diseases besides bacteria, such as cancer.

Examples of treatment enhancing agents (facilitating removal of persistent infection by microbial biofilms or antibiotic enhancers) include but are not limited to: D-amino acids, natural or synthetic proteolytic, polysaccharide, nucleic acids degradation enzymes and/or active domain fragments, antibody against biofilm structure stabilizers such as DNABII family member proteins (such as HU and IHF), or elements that can inhibit or breakdown such biofilm structure stabilizers; beta-lactamase inhibitors such as Clavulanic acid, sulbactam, tazobactam, Penicillin acid derivatives such as penicillin sulphones, penam sulphones, penems, Carbapenems, Monobactams, Cephalosporin-Based Inhibitors such as sodium salt of 7-[(Z)-(tertbutoxycarbonyl) methylene]cephalosporin acid sulphone, phosphonates and boronate inhibitors, BLIP (a 165-amino acid protein composed of two tandemly repeated domains that in the co-crystal BLIP-TEM) and derivatives, etc.

Disclosed herein, is antibiotic dosage impact on the growth of antibiotic resistant bacterial strains using antibiotics.

Disclosed herein, is also a previous study conducted in inventor's lab (under the inventor's direction) which illustrated the effect of the mode of administration of an antibiotic on antibiotic resistant bacteria colonizing the gastrointestinal tract and excreted into the environment. Effective dosage distribution through intravenous administration of an antibiotic generates fewer antibiotic gene pools than oral delivery of the antibiotic. Antibiotic resistant bacteria introduced with food and environmental exposure can form stable populations in the gastrointestinal tract. Only a percentage of the antibiotics administered orally will be absorbed and serve as effective role of inhibiting/killing the target organism(s).

Similar approaches such as patching, when locally applied drugs including but not limiting to modified antibiotic conjugates as described, can have much higher local dosage but less systematic impact on the whole body and microbiota than orally delivery, can effectively treat local infections including by antibiotic resistant bacteria.

It will be readily apparent to those skilled in the art that other suitable modifications and adaptations of the methods of the present disclosure described herein are obvious and may be made using suitable equivalents without departing from the scope of the present disclosure or the embodiments disclosed herein. Having now described the present disclosure in detail, the same will be more clearly understood by reference to the following example which is included for purposes of illustration only and not intended to limit the scope of the present disclosure. The disclosures of all journal references, U.S. patents and publications referred to herein are hereby incorporated by reference in their entireties.

### EXAMPLES

Study 1. The variability of antibiotic resistant (ART) bacteria in responding to drug treatment was evaluated using tetracycline resistant isolates in brain heart infusion (BHI) broth. The study involved tetracycline resistant isolates Plesiomonas shigelloides 17iT21, with minimum inhibition concentration (MIC, for tetracycline) of 64 μg/ml, Enterococcus sp. 17fT4, with MIC of 128 μg/ml, Enterococcus sp. 18fT3, with MIC of 512 μg/ml. All strains were isolated from aquaculture environment. The bacteria in different titer were grown in BHI with different concentration of tetracycline conjugated to antibody against Enterococcus. At tetracycline concentration below 64 μg/ml, Plesiomonas shigelloides 17iT21 grew in BHI with enterococcal antibody(EAB) conjugated tetracycline through the biotin-avidin-biotin link. However, at the same tetracycline concentration, both Enterococcus 17iT4 (MIC 128 μg/ml) and 181T3 (MIC 512 μg/ml), which should be more resistant to tetracycline than the Plesiomonas shigelloides 17iT21 (MIC 64 μg/ml), exhibited inhibition to growth by the EAB-conjugated tetracycline. The inhibition is more obvious in Enterococcus 17iT4, which has a lower MIC level between the two enterococcal strains.

Materials and Methods

Bacteria Cultivation

Plesiomonas shigelloides 17iT21, Enterococcus sp. 17fT4, and Enterococcus sp. 18fT3 were grown in BHI broth for overnight. Portions of the cells were diluted in BHI for 10⁻², 10⁻⁴, and 10⁻⁶ fold. Ten μl of the original or diluted cells were inoculated into 1 ml of 0.5×BHI containing 1× or 0.1×EAB conjugated tetracycline. The cultures were incubated at 30° C. for 16 hrs.

Conjugated Antibiotic Preparation

Approximately 3 mg of tetracycline were biotinylated using 2 mg of NHS-biotin reagent as instructed by the manufacturer. The mixture was dialyzed for 3 hrs and mixed with equal amount of EAB, followed by mixing with avidin in PBS buffer for 30 min. The EAB conjugated tetracycline solution was filter-sterilized and mixed with BHI with proper concentration. The broth is ready for bacteria inoculation.

Results

Plesiomonas shigelloides 17iT21 cells inoculated with 1% of overnight culture exhibited growth in both 1× and 0.1×EAB conjugated Tet with normal and heavy density, respectively, suggesting that the 1×EAB-Tet containing broth had less than 64 μg/ml Tet, and 0.1×EAB-Tet had less than 6.4 μg/ml Tet. No growth was observed in 1×EAB-Tet BHI-broth inoculated with 10⁻², 10⁻⁴, and 10⁻⁶ cells, but heavy growth was observed for all in 0.1×EAB-Tet BHI broth. The results suggested that cell concentration and antibiotic concentration both play a role in the effectiveness of inhibition of bacterial growth. Antibiotic at concentration even lower than the minimum inhibition concentration was still effective in inhibiting low density of resistant cells. Therefore antibiotic treatment is much more effective in early stage of infection, when cell numbers are relatively low.

Enterococcus sp. 17fT4 (Tet MIC 128 μg/ml), and Enterococcus sp. 18fT3 (Tet MIC 512 μg/ml) were grown in the same batch of EAB-Tet BHI broth as described above for Plesiomonas shigelloides 17iT21. 17fT4 exhibited reduced growth in 1×EAB-Tet BHI broth and normal growth in 0.1×EAB-Tet BHI broth inoculated with 1% of overnight culture, no growth in 1×EAB-Tet BHI broth and reduced growth in 0.1×EAB-Tet BHI broth inoculated with diluted cultures. The same trend was observed for 181T3, but the growth was relatively heavy than 17fT4, because it had high MIC so was more resistant to Tet.

By comparing the growth results between Plesiomonas shigelloides and Enterococcus sp. strains, it was obvious that although the Plesiomonas strain should be more sensitive to tetracycline because of lower MIC than that of the Enterococcus sp. strains, the EAB-Tet was much more effective in inhibiting the growth of Enterococcus strains with up to 8 fold MIC. The results suggested that the multiple components antibiotic formula can be rapidly assembled, and also can achieve effective inhibition to targeted resistant strains with less concentrated drug, likely due to improved local drug concentration. A net result of implementing the new drug formula can lead to less antibiotic selective pressure to the general microbiota and minimizing the development of drug resistant microbiota, as well as effective treatment of existing drug resistant bacteria. The impact of the biotinylation process on the specificity and activity of tetracycline needs to be further evaluated.

Study 2. The potency of foodborne antibiotic resistant (ART) bacteria in shaping microbial ecology in host gastrointestinal (GI) tract was evaluated using an animal model. Germ-free mice were inoculated with human associated microbiota and/or exposed to identified AR gene carriers (Tet^{r} Enterococcus faecium A21 and Streptococcus sp. A85) isolated from seafood products, with water and cheese as delivery vectors. The selected foodborne AR gene carriers successfully colonized and significantly amplified in the host GI tracts after a short exposure (2 days) in the presence of human associated microbiota, and persist in the absence of antibiotic selective pressure for up to 2 months. The findings suggest that many foodborne ART bacteria are capable of surviving the acidic environment in host digestive tract and colonizing host GI system.

The impact of different antibiotic delivery methods, exemplified by intravenous (IV) injection and oral feeding, on antibiotic resistance ecology in host GI tract was studied. Mice colonized by environment-associated microbiota were divided into oral group and IV group. Both groups of mice were administered with tetracycline hydrochloride (50 mg/kg body weight/day) for five days. Since only part of the antibiotic by oral delivery was absorbed by the host, the second round of the experiment used increased dosage for oral group and reduced dosage for IV group. Both tetracycline resistant population and tet gene pools (tet(L), tet(M)) in mouse fecal samples with equal dosage of antibiotic exposure were significantly amplified one day after initial antibiotic administration and maintained thereafter. Despite predominant ART bacteria population in mice fecal samples altered in a similar manner after initial antibiotic exposure, detected tet gene pools in mouse fecal sample from the oral group were 10-100 times larger than that of the IV group, indicating that oral drug administration may have bigger impact on AR ecology in host GI tract than IV injection. The difference was even bigger when increased and reduced dosage was applied to oral administration and IV injection, respectively (Ph.D. dissertation, Zhang, 2011). The study concluded that at least in the case of tetracycline, oral administration may have bigger impact on the development of ART microbiota in host GI tract than IV injection. The results indicated a possible major contributor for the rapid emergence of ART bacteria, and potential intervention point for effective mitigation by 1) reducing the exposure of the normal microbiota to antibiotics, 2) increasing the local dosage to targets.

Materials and Methods

Mouse Preparation

Animal protocol 2009A0167 and amendments (The Ohio State University) was followed throughout the study. Germ-free mice (known to be previously colonized with uncultivable Bacillus sp.) were kept in sterile passive vented cages and grouped as three mice per cage. Two groups were introduced as 1) one cage of control group, 2) two cages of mice fed on Enterococcus faecalis G37 (Em^{r} Tet^{s}) previously isolated from infant feces. E. faecalis strain was used to colonize germ-free mice GI tract before exposed to foodborne ART bacteria. The strain was inoculated into feeding water as 4×10⁴CFU/ml (final concentration) and the feeding water is the only water source to mice in the study. Mice were exposed to such feeding water for two days before the strain was withdrawn from the water source.

Two previously identified tetracycline resistant bacterial strains isolated from cooked shrimp samples were used to feed mice after initial colonization. Streptococcus sp. A85 (tet(S)) and Enteroccus faecium A21 (tet(S), tet/L), tet(M)) containing plasmid encoded tetracycline resistance genes were delivered in two independent pathways: 1) the strains were inoculated into feeding water with 10⁵ CFU/ml and renewed every day; 2) the strains were inoculated into sterilized cheddar cheese with 10⁵ to 10⁶ CFU/g and renewed every day. In both cases, the exposure lasted three continuous days before mice fecal samples were collected and finally analyzed.

After the exposure to foodborne ART bacteria through feeding water and/or cheese, ART bacteria were withdrawn from feeding and mice were fed on regular diet (treated with Gamma irradiation) exclusively. At the meantime, mice fecal samples were collected every other week to examine the presence of previously consumed ART bacteria.

Germ-free mice transferred into vented cages and exposed to environmental contamination for one year were observed to have developed cultivable ART bacteria in GI tract system. These mice were used in the antibiotic administration study.

Fecal Bacteria Enumeration

Fresh mouse fecal sample was homogenized in saline with a W/N ratio of 1 to 20 to 1 to 40. The liquid became zero dilution for plating. The liquid was then subjected to serial dilution and plated on Columbia Blood Agar (CBA) Base supplemented with 5% sheep blood. All media contained 100 μg/ml cycloheximide. Selective antibiotics included tetracycline (16 μg/ml), erythromycin (100 μg/ml), sulfamethoxazole/trimethoprim (152 μg/m1, 8 μg/ml respectively) and cefotaxime (4 μg/ml). 100 III of selected dilution was spread on agar medium and the plates were incubated in BD GasPak™ 150 anaerobic system with three BD GasPak™ EZ Anaerobe Sachets at 37° C. for 48 h.

Antibiotic Administration

Mice in oral group were fed with 0.2 ml 5 mg/ml tetracycline hydrochloride at 50 mg/kg body weight per day. Feeding was performed with 20G animal feeding gavage. Mice in IV group were restrained on TV-150 tail vein restrainer (Braintree Scientific) and injected with BD 0.5 ml insulin syringe. A total of 0.2 ml 5 mg/ml tetracycline hydrochloride was injected into mouse tail vein once a day. Mice from both groups were administered with tetracycline hydrochloride for five days. Another comparison group between oral and IV injection utilized 100 mg/kg body weight per day as oral dosage and 10 mg/kg body weight per day as IV dosage. Mouse fecal sample were collected on day 1, 2, 5, 7 to examine ART bacteria and AR gene pool.

DNA Extraction from Mouse Fecal Sample

The procedure described by Li and Wang (Journal Food Prot, 2010), and Zhang et al (Appl Environ Microbiol, 2011) was followed to extract DNA from bacterial isolates as amplification templates for conventional PCR. For real-time quantitative PCR and DGGE analyses, the DNA templates from mouse fecal samples were extracted according to Yu and Morrison.

Examination of AR Gene Pool by Real-Time Quantitative PCR

Various AR gene pools, including tet(S), tet(L), tet(M), ermB, sull, sul2, bla_{TEM}. were examined using total DNA extract from mouse fecal samples. Real-time quantitative PCR was performed on a CFX real-time system (Bio-rad).

Denaturing Gradient Gel Electrophoresis Analysis on Plate DNA

DGGE analysis was performed as described by Li and Wang (Journal Food Prot, 2010) and Zhang et al (Appl Environ Microbiol, 2011). All recovered DNA bands were sequenced at Plant Microbe Genomic Facility at The Ohio State University.

Identification of AR Gene Carriers in Mouse Fecal Sample

The same procedure described in the above literatures was adopted. All AR gene carriers were identified at Plant Microbe Genomic Facility at The Ohio State University.

Evaluation of the Effect of Antibiotics in Mouse Tissue

One cage of mice (three) was fed with tetracycline hydrochloride and the other cage of mice (four) were IV injected with the same amount of antibiotic. Two hours after the exposure, these two cages of mice, together with one mouse in the control group, were sacrificed to collect muscle, cecum and colon. Mouse muscle, cecum and colon were first soaked into liquid nitrogen and then stored in −80° C.

Results

Establishment of Human Associated Flora and Foodborne ART Bacteria in Host GI Tract

After being exposed to Erm^{r} E. faecalis G37 strain for two days (in water), the strain was detected in mice fecal sample in 2×10⁹ CFU/g on the third day and 5×10¹² CFU/g in a week. Similarly, with the presence of previously inoculated Enterococcus faecalis G37, foodborne Enterococcus faecium A21 and Streptococcus sp. A85 were detected in mice feces in 10⁹ CFU/g and thereafter. These strains were still detected in mice fecal samples two months after the exposure.

On the other hand, though the E. faecium A21 strain colonized mice GI tract well whether through feeding water or cheese inoculation, the Streptococcus sp. A85 strain only colonized mice GI tract well when using cheese as delivery vector, as the strain died out in a couple of hours in water. Therefore food delivery method had direct impact on the efficacy of inoculation.

No transconjugants were detected from mouse fecal sample between foodborne ART bacteria strains and previously colonized E. faecalis G37, though both foodborne strains contained identified tet genes on mobile gene elements.

Change of ART Bacterial Population in Mouse GI Tract Upon Antibiotic Exposure

The application of tetracycline hydrochloride did not cause significant impact on total bacteria count in mouse feces. However, it was observed that Tet^{r} population significantly increased after the exposure.

Change of AR Gene Pools in Mouse GI Tract Upon Exposure to Antibiotics

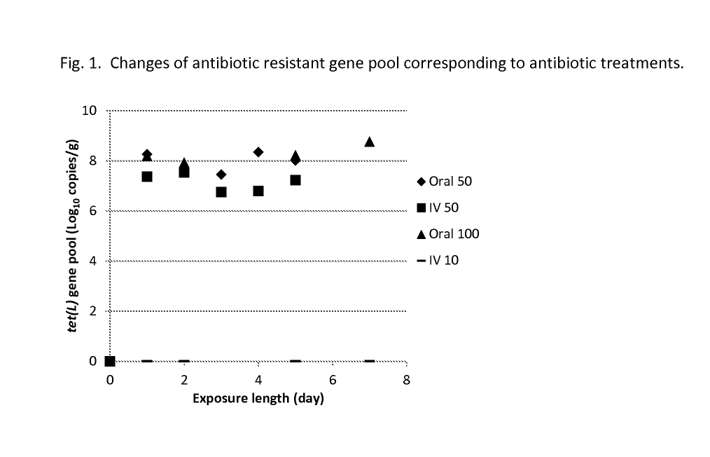

Among seven AR genes examined, tet(S), sul1, sul2, bla_{TEM} were not detected in mouse fecal samples, regardless whether the mouse had been treated with tetracycline hydrochloride or not. The sizes of te(L), tet(M) gene poolss dramatically increased in mouse fecal samples after antibiotic exposure (FIG. 1). Oral administration caused even greater increase (10-100 times) of tet(L) and tet(M) gene pool than IV injection throughout the exposure. A much smaller AR gene pools were found in mice group with reduced Tet injection dosage.

Change of profile of predominant ART bacteria after antibiotic treatment Predominant ART bacteria in mouse GI tract were examined before and after antibiotic exposure. Major population switch existed right after antibiotic administration. For example, Streptococcus sp was found to be predominant in Tet^{r} population; however, one day after mouse was exposed to tetracycline hydrochloride, Enterococcus sp. started to thrive in mouse GI tract. Similarly, Enterobacter sp. was found to be predominant bacteria in Erm^{r} population and after antibiotic exposure, Enterococcus sp. was detected in predominant population. Though an abrupt change of constitution of predominant ART population was observed right after antibiotic administration, the composition of Tet^{r}, Erm^{r} Sul^{r} and Ctx^{r} population remained relatively stable throughout the entire antibiotic treatment period in oral and IV groups. There was no difference observed in terms of the effect on the profile of ART bacteria in mouse GI tract between oral delivery and IV injection.

Profile of AR gene carriers in mouse GI tract before and after antibiotic administration.

As tet(L), tet(M) and ermB gene pools were detected in total DNA from mouse fecal samples, Tet^{r} and Erm^{r} isolates in fecal samples from mice before antibiotic administration, after 5 days continuous exposure via oral delivery or IV injection were randomly selected and screened for these AR genes. It was observed that various Enterococcus species and subspecies served as AR gene carriers in mouse GI tract. Prevalence of AR gene carriers in corresponding ART population changed upon antibiotic exposure.

What is claimed is:

1. A method for targeting an antibiotic to a site, in a subject, the method comprising administering to the subject an antibiotic-ligand conjugate, the conjugate comprising an antibiotic modified to include a first binding site and a ligand modified to include a second binding site that binds to the first binding site, wherein the ligand targets the antibiotic to the site.

2. The method of claim 1, wherein the ligand is an antibody or adjunct.

3. The method of claim 1 or 2, wherein the site comprises a cell, tissue, organ, organism, microorganism, infectious agent, bacterium, virus, fungus, disease site, immune response factor, or any combination thereof.

4. The method of any one of claims 1 to 3, further comprising the step of contacting the antibiotic and the ligand to form the antibiotic-ligand conjugate prior to administration to the subject.

5. The method of claim 4, wherein the conjugate is administered to the subject within two months of contacting the ligand and the antibiotic.

6. The method of claim 4, wherein the conjugate is administered to the subject within 1 week of contacting the ligand and the antibiotic.

7. The method of claim 4, wherein the conjugate is administered to the subject within 48 hours contacting the ligand and the antibiotic.

8. The method of claim 4, wherein the conjugate is administered to the subject within 12 hours contacting the ligand and the antibiotic.

9. The method of claim 4, wherein the conjugate is administered to the subject within 1 hour of contacting the ligand and the antibiotic.

10. The method of any preceding claim, wherein the first binding site or the second binding site, or both the first and second binding sites comprise a component selected from the group consisting of biotin (avidin, streptavidin), GSTs and glutathione, poly His tag, Ni or cobalt or affinity agent(s), natural or synthesized FLAG-tag or FLAG octapeptide, HA (hemagglutinin) tag, myc-tag (N-EQKLISEEDL-C) and antibody (such as 9E10), drug conjugates, multi-specific antibodies, Fc engineered antibodies, scFv fused receptors, peptide and engineered antigens and antibodies, site-specific antibodies and biomarkers; anti-DDDDK tag, anti-R Phycoerythrin, anti-VSV-G tag, anti-digoxigenin, anti-biotin, anti-FITC, anti-poly (5×, 6×, 8× etc) His tag, anti-V5 tag, peptides, nucleotides, small molecules with reactive groups, and any combination thereof.

11. A method for formulating a targeted and personalized antibiotic treatment to a site, organism or host responsive target in a subject, the method comprising formulating, combining and administering to the subject an antibiotic-ligand conjugate, the conjugate comprising an antibiotic modified to include a first binding site and a ligand modified to include a second binding site that binds to the first binding site, wherein the ligand targets the antibiotic to the site.

12. The method of claim 11, wherein the ligand is an antibody or adjunct.

13. The method of claim 11 or 12, wherein the site comprises a cell, tissue, organ, organism, microorganism, infectious agent, bacterium, virus, fungus, disease site, immune response factor, or any combination thereof.

14. The method of any one of claims 11 to 13, further comprising the step of contacting the antibiotic and the ligand to form the antibiotic-ligand conjugate prior to administration to the subject.

15. The method of claim 14, wherein the conjugate is administered to the subject within two months of contacting the ligand and the antibiotic.

16. The method of claim 14, wherein the conjugate is administered to the subject within 1 week of contacting the ligand and the antibiotic.

17. The method of claim 14, wherein the conjugate is administered to the subject within 48 hours contacting the ligand and the antibiotic.

18. The method of claim 14, wherein the conjugate is administered to the subject within 12 hours contacting the ligand and the antibiotic.

19. The method of claim 14, wherein the conjugate is administered to the subject within 1 hour of contacting the ligand and the antibiotic.

20. The method of any preceding claim, wherein the first binding site or the second binding site, or both the first and second binding sites comprise a component selected from the group consisting of biotin (avidin, streptavidin), GSTs and glutathione, poly His tag, Ni or cobalt or affinity agent(s), natural or synthesized FLAG-tag or FLAG octapeptide, HA (hemagglutinin) tag, myc-tag (N-EQKLISEEDL-C) and antibody (such as 9E10), drug conjugates, multi-specific antibodies, Fc engineered antibodies, scFv fused receptors, peptide and engineered antigens and antibodies, site-specific antibodies and biomarkers; anti-DDDDK tag, anti-R Phycoerythrin, anti-VSV-G tag, anti-digoxigenin, anti-biotin, anti-FITC, anti-poly (5×, 6×, 8× etc) His tag, anti-V5 tag, peptides, nucleotides, small molecules with reactive groups, and any combination thereof.
